(19) United States
(12) Patent Application Publication
(10) Pub. No.: US 2021/0088511 A1
(43) Pub. Date: Mar. 25, 2021

(54) METHODS AND COMPOSITIONS FOR DETECTION AND ANALYSIS OF ANALYTES

(71) Applicant: Roche Diagnostics Operations, Inc., Indianapolis, IN (US)

(72) Inventors: Peter CRISALLI, Mountain View, CA (US); Dmitriy GREMYACHINSKIY, Sunnyvale, CA (US); Dieter HEINDL, Muenchen (DE); Hannes KUCHELMEISTER, Muenchen (DE); Michael SCHRAEML, Penzberg (DE); Andrew TRANS, Mountain View, CA (US)

(73) Assignee: Roche Diagnostics Operations, Inc., Indianapolis, IN (US)

(21) Appl. No.: 17/039,245

(22) Filed: Sep. 30, 2020

Related U.S. Application Data
(63) Continuation of application No. PCT/EP2019/059363, filed on Apr. 12, 2019.
(60) Provisional application No. 62/657,394, filed on Apr. 13, 2018.

(51) Int. Cl. G01N 33/543; G01N 33/487

(52) U.S. Cl. CPC G01N 33/5438; G01N 33/48721

(57) ABSTRACT

Provided are nanopore-based methods, compositions, and systems for assessing analyte-ligand interactions and analyte concentration in a fluid solution. The compositions include an analyte detection complex that is associated with a nanopore to form a nanopore assembly, the analyte detection complex including an analyte ligand. As a first voltage is applied across the nanopore assembly, the analyte ligand is presented to an analyte in the solution. As a second voltage that is opposite in polarity to the first voltage is applied across the nanopore assembly, the analyte binds to the analyte. By comparing the total number of analyte-ligand binding pairs to a control binding count, the concentration of the analyte can be determined. In other examples, further increasing the second voltage can result in dissociation of the analyte-ligand pair, from which a dissociation voltage—and hence a dissociation constant—can be determined.

### CROSSREFERENCE TO RELATED APPLICATIONS

This application is a continuation application of PCT/EP2019/059363, filed Apr. 12, 2019, which claims the benefit of U.S. Application No. 62/657,394, filed Apr. 13, 2018, each of which is incorporated herein by reference in its entirety.

### TECHNICAL FIELD

The present disclosure relates generally to methods, compositions, and systems for detecting a target analyte, and more particularly to methods, compositions, and systems for determining the concentration of an analyte and for assessing analyte-ligand interactions using a biochip.

Provided are nanopore-based methods, compositions, and systems for assessing analyte-ligand interactions and analyte concentration in a fluid solution. The compositions include an analyte detection complex that is associated with a nanopore to form a nanopore assembly, the analyte detection complex including an analyte ligand. As a first voltage is applied across the nanopore assembly, the analyte ligand is presented to an analyte in the solution. As a second voltage that is opposite in polarity to the first voltage is applied across the nanopore assembly, the analyte binds to the analyte. By comparing the total number of analyte-ligand binding pairs to a control binding count, the concentration of the analyte can be determined. In other examples, further increasing the second voltage can result in dissociation of the analyte-ligand pair, from which a dissociation voltage—and hence a dissociation constant—can be determined.

### BACKGROUND OF THE INVENTION

Biologically active components, such as small molecules, proteins, antigens, immunoglobulins, and nucleic acids, are involved in numerous biological processes and functions. Hence, any disturbance in the level of such components can lead to disease or accelerate the disease process. For this reason, much effort has been expended in developing reliable methods to rapidly detect and identify biologically active components for use in patient diagnostics and treatment. For example, detecting a protein or small molecule in a blood or urine sample can be used to assess a patient's metabolic state. Similarly, detection of an antigen in a blood or urine sample can be used to identify pathogens to which a patient has been exposed, thus facilitating an appropriate treatment. It is further beneficial to be able to determine the concentration of an analyte in solution. For example, determining the concentration of a blood or urine component can allow the component to be compared to a reference value, thus facilitating further evaluation of a patient's health status.

Nevertheless, while numerous detection and identification methods are available, many are expensive and can be rather time consuming. For example, many diagnostic tests can take several days to complete and require significant laboratory resources. And in some cases, diagnostic delays can negatively impact patient care, such as in the analysis of markers associated with myocardial infarction. Further, the complexity of many diagnostic tests aimed at identifying biologically active components lends itself to errors, thus reducing accuracy. And, many detection and identification methods can only analyze one or a few biological active components at a time, and they cannot determine concentration of a given component of the test sample.

In addition to identifying biologically active components in a test sample, it is also desirable to screen biological samples for novel binding pairs, such as small molecule-protein binding pairs or protein-protein binding pairs. For example, determining that a particular protein binds a small molecule may lead to the development of the small molecule as new a therapeutic drug or diagnostic reagent. Likewise, the identification of a new protein-protein interaction may lead to the development of a new drugs or diagnostic reagents. But while many traditional methods are available to examine interactions between different biologically active components, such methods are often designed to examine one or a few candidate binding pairs at a time. Such methods are also costly and can be time consuming.

Hence, what is needed are additional methods, compositions, and systems that can rapidly detect and identify biologically active components, especially in an efficient and cost-effect manner. Also needed are methods, compositions, and systems that can assay multiple biologically active components at the same time, thus reducing costs. Further, methods, compositions, and systems are needed to determine the concentration of a biologically active component in a fluid solution. Also needed are rapid and cost-effective methods to assess binding interactions between biologically active components, thereby further facilitating the development of new drugs and therapeutic approaches.

### SUMMARY OF THE INVENTION

In certain example aspects, provided is an analyte detection complex that includes an analyte ligand, a threading element, a signal element, and an anchoring tag. The analyte ligand is located on a proximal end of the analyte detection complex while the signal element is associated within the threading element. The analyte detection complex can also include an anchoring tag on the distal end of the threading element. In certain example aspects, the analyte detection complex also includes a second signaling element.

In certain example aspects, provided is nanopore assembly that includes an analyte detection complex. For example, the nanopore assembly can be heptameric alpha-hemolysin nanopore assembly. The analyte detection complex, for example, is threaded through the nanopore to form a nanopore assembly.

In certain example aspects, provided is a method for assessing binding strength between an analyte and an analyte ligand. The method includes providing, in the presence of a first voltage, a chip that includes a nanopore assembly as described herein. The nanopore assembly, for example, is disposed within a membrane. A sensing electrode is positioned adjacent or in proximity to the membrane. The method also includes contacting the chip with a fluid solution that includes the analyte, the analyte having a binding affinity for the analyte ligand of the analyte detection complex. Thereafter, an incrementally increased second voltage is applied across the membrane, the second voltage being opposite in polarity to the first voltage. In response to applying the incrementally increased second voltage across the membrane, a binding signal is determined with the aid of the sensing electrode, the binding signal providing an indication that the analyte is bound to the analyte ligand. And as the second voltage is further increased, a dissociation signal is determined with the aid of the sensing electrode, the dissociation signal providing an indication of the binding strength between the analyte and analyte ligand.

In certain example aspects, the method further includes using the sensing electrode to detect a threading signal, the threading signal providing an indication that the threading element is located within the pore of the nanopore assembly. In certain example aspects, the threading signal is compared to the binding signal. The comparison, for example, can provide the indication that the analyte is bound to the analyte ligand.

In certain example aspects, the method further includes determining, from the dissociation signal, a dissociation voltage associated with dissociation of the analyte from the analyte ligand. By comparing the determined dissociation voltage with a reference dissociation voltage, a dissociation constant for the analyte and analyte ligand binding pair can be determined.

In certain example aspects, provided is a method of determining the concentration of an analyte in a fluid solution. The method includes, for example, providing, in the presence of a first voltage, a chip including multiple nanopore assemblies as described herein. The nanopore assemblies, for example, are disposed within a membrane, and at least a first subset of the nanopore assemblies includes a first analyte ligand. The method also includes positioning multiple sensing electrodes adjacent or in proximity to the membrane and contacting the chip with a fluid solution. The fluid solution includes a first analyte, the first analyte having a binding affinity to the first analyte ligand. With the aid of the sensing electrodes and a computer processor, a binding count is then determined. The binding count, for example, provides an indication of the number of binding interactions between the first analyte ligand and the first analyte. By then comparing the determined binding count to a reference count, a concentration of the analyte in the fluid solution can be determined.

In certain example aspects, determining the binding count includes using the sensing electrodes to determine, for each nanopore assembly of the first subset of nanopore assemblies, a threading signal. The threading signal, for example, provides an indication that the threading element is located within the nanopore of the nanopore assembly. Thereafter, an incrementally increased second voltage is applied across the membrane, the second voltage having a polarity that is opposite in to the first voltage. In response to applying the incrementally increased second voltage across the membrane, the sensing electrodes are used to determine—for each nanopore assembly of the first subset of nanopore assemblies—a binding signal. The method then includes comparing, for each nanopore assembly of the first subset of nanopore assemblies, the determined threading signal with the determined binding signal. The comparison, for example, provides an indication that the first analyte is bound to the first analyte ligand. From the comparison of each of the determined threading signals with the determined binding signals, a total number of indications that the first analyte is bound to the first analyte ligand can be determined, the total number of indications corresponding to the binding count. In certain example aspects, the binding count is compared to a reference binding count.

These and other aspects, objects, features and advantages of the example embodiments will become apparent to those having ordinary skill in the art upon consideration of the following detailed description of illustrated example embodiments.

### DETAILED DESCRIPTION OF THE INVENTION

The embodiments described herein can be understood more readily by reference to the following detailed description, examples, and claims, and their previous and following description. Before the present system, devices, compositions and/or methods are disclosed and described, it is to be understood that the embodiments described herein are not limited to the specific systems, devices, and/or compositions methods disclosed unless otherwise specified, as such can, of course, vary. It is also to be understood that the terminology used herein is for the purpose of describing particular aspects only and is not intended to be limiting.

Further, the following description is provided as an enabling teaching of the various embodiments in their best, currently known aspect. Those skilled in the relevant art will recognize that many changes can be made to the aspects described, while still obtaining the beneficial results of this disclosure. It will also be apparent that some of the desired benefits of the present invention can be obtained by selecting some of the features of the various embodiments without utilizing other features. Accordingly, those who work in the art will recognize that many modifications and adaptations to the various embodiments described herein are possible and can even be desirable in certain circumstances and are a part of the present disclosure. Thus, the following description is provided as illustrative of the principles of the embodiments described herein and not in limitation thereof.

### OVERVIEW

As described herein, provided are nanopore-based methods, compositions, and systems for determining the concentration of an analyte in a fluid solution. Also provided are nanopore-based methods, compositions, and systems for assessing analyte-ligand binding interactions in a fluid solution. The compositions include, for example, an analyte detection complex that is associated with a nanopore to from a nanopore assembly, the analyte detection complex including an analyte ligand. As a first voltage is applied across a membrane including the nanopore assembly, the analyte ligand is presented to the cis side of the nanopore where it can bind an analyte in the fluid solution. As a second voltage that is opposite in polarity to the initial voltage is applied across the membrane, a signal indicating binding between the analyte and the analyte ligand can be determined. By determining the total number of analyte-ligand binding pairs across multiple nanopore assemblies—and comparing that value a known reference value—the concentration of the analyte in the solution can be determined. In other examples, further increasing the second voltage can result in dissociation of the analyte-ligand pair, from which a dissociation voltage—and hence a dissociation constant—can be determined.

More particularly, the analyte ligand of the analyte detection complex can be any ligand that targets an analyte. For example, the analyte ligand can be an antibody or functional fragment thereof that targets a specific antigen, thus providing an immunoassay-type method to identify the antigen. In certain examples, the analyte is a blood antigen or other biological fluid antigen. In other examples, the analyte is a polypeptide, amino acid, polynucleotide, carbohydrate, or small molecule organic compound or inorganic compound to which the analyte ligand of the analyte detection complex has affinity.

In addition to the analyte ligand, the analyte detection complex includes a threading element that is joined to the analyte ligand. The threading element, for example, can be a single or double stranded nucleic acid sequence or other molecular polymer that can be threaded through the pore of a nanopore. The analyte ligand is joined to the proximal end of the threading element, while the distal end of the threading element is associated with an anchoring tag. The anchoring tag, for example, can be used to prevent the distal end of the threading element from moving through the nanopore assembly to the cis side of the nanopore assembly. Associated with the threading element is one or more signal elements that can be used to vary the electronic signal through the pore. The signal element of the analyte detection complex can be any entity that can be positioned within the pore of a nanopore assembly, such as an oligonucleotide, a peptide, or polymer. In certain examples, one or more signal elements can be used to determine the position of the threading element within the pore of the nanopore assembly.

When assembled into a membrane of a chip, a nanopore assembly that includes the analyte detection complex as described herein can be used to assess the binding interactions between an analyte and an analyte ligand. The nanopore, for example, can be any protein nanopore, such as an alpha-hemolysin (α-HL) nanopore, OmpG nanopore, or other protein nanopores. Without wishing to be bound by any particular theory, when the first voltage is applied across a membrane including the nanopore assembly, the proximal end of the analyte detection complex threads through the pore, thereby locating the threading element—and its one or more signal elements—within the pore. Further, with the analyte detection complex threading through the pore, the analyte ligand of the analyte detection complex can be presented to the cis side of the nanopore assembly where it can interact with (and bind) an analyte. In certain examples, an electrode associated with the nanopore assembly can be used to determine a threading signal corresponding to the presence of the threading element in the pore. For example, in response to the first voltage being applied across the membrane, a first signal element associated with the threading element can locate within the pore in such a way that positioning of the threading element within the pore can be determined via the sensing electrode.

Once the threading element is located within the pore of the nanopore assembly—and the analyte ligand has had a chance to bind the analyte—a second voltage having a polarity opposite to the first voltage can be incrementally applied across the membrane. The second voltage, for example, operates to pull the analyte detection complex towards the trans side of the nanopore assembly. Without wishing to be bound by any particular theory, in the absence of the analyte the pulling force pulls the analyte detection complex through the pore to the trans side of the pore. But in the presence of the analyte, the binding of the analyte ligand to the analyte on the cis side of the nanopore assembly prevents the analyte detection complex from moving through the pore to the trans side of the nanopore assembly. In certain examples, the pulling force arising from the second voltage positions a second signal element within the pore so that a binding signal can be determined from the electrode associated with the nanopore assembly. The binding signal, for example, can provide an indication that the analyte is bound to the analyte ligand.

To assess binding interactions between the analyte and the analyte ligand, such as the strength of the binding, the second voltage can be further increased until a dissociation signal is obtained from the nanopore assembly via the associated electrode. The dissociation signal, for example, corresponds to the point where the increased voltage forces the analyte to separate from the analyte ligand, thus allowing the analyte detection complex to be pulled through the pore to the trans side of the membrane. Based on the dissociation signal, a dissociation voltage can be determined that corresponds to the voltage at which the dissociation between analyte and the analyte ligand occurs. In certain examples, the dissociation voltage can be compared to one or more reference voltages of a known analyte-ligand pair, thus allowing determination of a dissociation constant for the analyte and the analyte ligand.

In certain examples, the binding between the analyte and analyte ligand can be so strong that the analyte does not separate from the analyte ligand. Rather, the analyte remains bound to the analyte ligand even when the second voltage is further increased. In such examples, when multiple different analytes are assessed for their binding properties to different analyte ligands, the analytes with the strongest binding properties can be easily identified. In other examples, multiple analytes are analyzed to determine their relative binding strengths to one or more analyte ligands. For example, binding strengths may be determined as weak, strong, or very strong for different analyte-ligand interactions on the same chip.

In certain examples, the methods, compositions, and systems described herein can also be used to determine the concentration of a test analyte in a fluid solution. For example, multiple nanopore assemblies can be formed on a chip in the presence of the first voltage as described herein, thereby presenting multiple analyte ligands to the test analyte on the cis side of each nanopore assembly. A fluid sample can then be applied to the cis side of the membrane. When the test analyte is present in the fluid sample, the test analyte can bind the analyte ligand. Thereafter, the second voltage opposite in polarity to the first voltage can be incrementally applied across the membrane as described herein, pulling each analyte detection complex towards the trans side of the membrane. But as described herein, binding of an analyte to the analyte ligand can prevent the analyte detection complex from moving through the pore to the trans side of the pore. Further, movement of a signaling element into the pore of the nanopore assembly can allow the determination of a binding signal.

By counting the number of binding singles, a binding count that corresponds to the total number of analyte-ligand interactions—and hence the number of test analytes bound—can be determined. The binding count can then be compared to a reference count to determine the concentration of the test analyte in solution. For example, a known amount of a second analyte can be included in the fluid sample as a control, and the number of bindings between the second analyte and a second analyte ligand can be determined as described herein as the reference count. The binding count can then be compared to the reference count to determine the concentration of the tests analyte.

In certain example embodiments, the methods described herein can be repeated on a chip to increase the confidence of the assessment. For example, if multiple nanopore assemblies are used to assess binding strength between different analyte-ligand pairs, the second voltage can be increased until the ligand-pairs dissociate. Then, the first voltage can be re-applied to re-localize the analyte detection complexes within the pores and to allow analyte-ligand binding. Following binding, the second voltage (opposite in polarity to the initial voltage) can be re-applied until the analyte-ligand pairs dissociate, thereby providing additional measurements of binding strength as described herein. Similarly, for concentration determinations, once binding counts are determined for analyte-ligand pairs as described herein, the second voltage can be applied to force dissociation of the analyte-ligand binding pairs. The steps of the concentration determination can be repeated to re-determine the concentration of the analyte. In certain example embodiments, the methods are repeated multiple times to further increase confidence level of the binding strength and/or concentration assessment.

### SUMMARY OF TERMS

The invention will now be described in detail by way of reference only using the following definitions and examples. Unless defined otherwise herein, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. It is to be understood that this invention is not limited to the particular methodology, protocols, and reagents described, as these may vary.

The headings provided herein are not limitations of the various aspects or embodiments of the invention which can be had by reference to the specification as a whole. Accordingly, the terms defined immediately below are more fully defined by reference to the specification as a whole.

As used herein, the singular forms “a”, “an” and “the” include plural referents unless the context clearly dictates otherwise.

Ranges or values can be expressed herein as from “about” one particular value, and/or to “about” another particular value. When such a range is expressed, another aspect includes from the one particular value of the range and/or to the other particular value of the range. It will be further understood that the endpoints of each of the ranges are significant both in relation to the other endpoint, and independently of the other endpoint. Similarly, when values are expressed as approximations, by use of the antecedent “about”, it will be understood that the particular value forms another aspect. In certain example embodiments, the term “about” is understood as within a range of normal tolerance in the art for a given measurement, for example, such as within 2 standard deviations of the mean. In certain example embodiments, depending on the measurement “about” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about. Further, terms used herein such as “example”, “exemplary”, or “exemplified”, are not meant to show preference, but rather to explain that the aspect discussed thereafter is merely one example of the aspect presented.

As used herein, the term “antibody” broadly refers to any immunoglobulin (Ig) molecule comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody entities are known in the art. A functional fragment of the antibody, for example, includes any portion of the antibody that, when separated from the antibody as whole retains the ability to bind or partially bind the antigen to which the antibody is directed. A “nanobody”, for example, is a single-domain antibody fragment.

As used herein, the term “amino acid” is an organic compound containing an amino group and a carboxylic acid group. A peptide or polypeptide contains two or more amino acids. For purposes herein, amino acids include the twenty naturally-occurring amino acids, non-natural amino acids and amino acid analogs (i.e., amino acids wherein the α-carbon has a side chain).

As used herein, “polypeptide” as used herein, refers to any polymeric chain of amino acids. The terms “peptide” and “protein” are used interchangeably with the term polypeptide and also refer to a polymeric chain of amino acids. The term “polypeptide” encompasses native or artificial proteins, protein fragments and polypeptide analogs of a protein sequence. A polypeptide may be monomeric or polymeric, and may include a number of modifications. Generally, a peptide or polypeptide is greater than or equal to 2 amino acids in length, and generally less than or equal to 40 amino acids in length.

As used herein, “alpha-hemolysin”, “α-hemolysin”, “α-HL”, “α-HL”, and “hemolysin” are used interchangeably and refer to the monomeric protein that self-assembles into a heptameric water-filled transmembrane channel (i.e., nanopore). Depending on context, the term may also refer to the transmembrane channel formed by seven monomeric proteins. In certain example embodiments, the alpha-hemolysin is a “modified alpha-hemolysin”, meaning that alpha-hemolysin originated from another (i.e., parental) alpha-hemoly sin and contains one or more amino acid alterations (e.g., amino acid substitution, deletion, or insertion) compared to the parental alpha-hemolysin. In some example embodiments, a modified alpha-hemolysin of the invention is originated or modified from a naturally-occurring or wild-type alpha-hemolysin. In some example embodiments, a modified alpha-hemolysin is originated or modified from a recombinant or engineered alpha-hemolysin including, but not limited to, chimeric alpha-hemolysin, fusion alpha-hemolysin or another modified alpha-hemolysin. Typically, a modified alpha-hemolysin has at least one changed phenotype compared to the parental alpha-hemolysin. In certain example embodiments, the alpha-hemolysin arises from a “variant hemolysin gene” or is a “variant hemolysin”, which means, respectively, that the nucleic acid sequence of the alpha-hemolysin gene from Staphylococcus aureus has been altered by removing, adding, and/or manipulating the coding sequence or the amino acid sequence of the expressed protein has been modified consistent with the invention described herein.

As used herein, the term “analyte” or “target analyte” refers broadly to any compound, molecule, or other substance of interest to be detected, identified, or characterized. For example, the term “analyte” or “target analyte” includes any physiological molecule or agent of interest that is a specific substance or component that is being detected and/or measured. In certain example embodiments, the analyte is a physiological analyte of interest. Additionally or alternatively, the analyte can be a chemical that has a physiological action, for example, or a drug or pharmacological agent. Additionally or alternatively, the analyte or target analyte can be an environmental agent or other chemical agent or entity. The term “agent” is used herein to denote a chemical compound, a mixture of chemical compounds, a biological macromolecule, or an extract made from biological materials. For example, an agent can be a cytotoxic agent.

In certain examples embodiments, the example “analytes” or “target analytes” include toxins, organic compounds, proteins, peptides, microorganisms, amino acids, carbohydrates, nucleic acids, hormones, steroids, vitamins, drugs (including those administered for therapeutic purposes as well as those administered for illicit purposes), lipids, virus particles, and metabolites of or antibodies to any of the above substances. For example, such analytes can include ferritin; creatinine kinase MIB (CK-MIB); digoxin; phenytoin; phenobarbitol; carbamazepine; vancomycin; gentamycin; theophylline; valproic acid; quinidine; leutinizing hormone (LH); follicle stimulating hormone (FSH); estradiol, progesterone; IgE antibodies; vitamin B2 micro-globulin; glycated hemoglobin (Gly. Hb); cortisol; digitoxin; N-acetylprocainamide (NAPA); procainamide; antibodies to rubella, such as rubella-IgG and rubella-IgM; antibodies to toxoplasmosis, such as toxoplasmosis IgG (Toxo-IgG) and toxoplasmosis IgM (Toxo-IgM); testosterone; salicylates; acetaminophen; hepatitis B virus surface antigen (HBsAg); antibodies to hepatitis B core antigen, such as anti-hepatitis B core antigen IgG and IgM (Anti-HBC); human immune deficiency virus 1 and 2 (HTLV); hepatitis B e antigen (HBeAg); antibodies to hepatitis B e antigen (Anti-Hbe); thyroid stimulating hormone (TSH); thyroxine (T4); total triiodothyronin (Total T3); free triiodiothyronin (Free T3); carcinoembryoic antigen (CEA); and alpha fetal protein (AF); and drugs of abuse and controlled substances, including but not intended to be limited to, amphetamine; methamphetamine; barbituates such as amobarbital, seobarbital, pentobarbital, phenobarbital, and barbital; benzodiazepines such as librium and valium; cannabinoids such as hashish and marijuana; cocaine; fetanyl; LSD; methapualone; opiaets such as heroin, morphine, codine, hydromorphone, hydrocodone, methadone, oxycodone, oxymorphone and opium; phencyclidine; and propoxyhene. The term analyte also includes any antigenic substances, haptens, antibodies, macromolecules and combinations thereof.

Other example analytes or target analytes include, Folate, Folate RBC, Iron, Soluble transferrin receptor, Transferrin, Vitamin B12, Lactate Dehydrogenase, Bone Calcium, N-MID Osteocalcin, P1NP, Phosphorus, PTH, PTH (1-84), b-CrossLaps, Vitamin D, Cardiac Apolipoprotein Al, Apolipoprotein B, Cholesterol, CK, CK-MB, CK-MB (mass), CK-MB (mass) STAT, CRP hs, Cystatin C, D-Dimer, Cardiac Digitoxin, Digoxin, GDF-154, HDL Cholesterol direct, Homocysteine, Hydroxybutyrat Dehydrogenase, LDL Cholesterol direct, Lipoprotein (a), Myoglobin, Myoglobin STAT, NT-proBNP, NT-proBNP STAT, 1 Troponin I, 1 Troponin I STAT, Troponin T hs, Troponin T hs STAT, Coagulation AT III, D-Dimer, Drugs of Abuse Testing Amphetamines (Ecstasy), Benzodiazepines, Benzodiazepines (Serum), Cannabinoids, Cocaine, Ethanol, Methadone, Methadone metabolites (EDDP), Methaqualone, Opiates, Oxycodone, 3, Phencyclidine, Propoxyphene, amylase, ACTH, Anti-Tg, Anti-TPO, Anti-TSH-R, Calcitonin, Cortisol, C-Peptide, FT3, FT4, hGH, Hydroxybutyrate Dehydrogenase, IGF-14, Insulin, Lipase, PTH STAT, T3, T4, Thyreoglobulin (TG II), Thyreoglobulin confirmatory, TSH, T-uptake, Fertility Anti Muellerian Hormone, DHEA-S, Estradiol, FSH, hCG, hCG plus beta, LH, Progesterone, Prolactin, SHBG, Testosterone, Hepatology AFP, Alkaline phosphatase (IFCC), Alkaline phosphatase (opt.), 3, ALT/GPT with Pyp, ALT/GPT without Pyp, Ammonia, Anti-HCV, AST/GOT with Pyp, AST/GOT without Pyp, Bilirubin—direct, —total, Cholinesterase Acetyl, 3 Cholinesterase Butyryl, Gamma Glutamyl Transferase, Glutamate Dehydrogenase, HBeAg, HBsAg, Lactate Dehydrogenase, Infectious Diseases Anti-HAV, Anti-HAV IgM, Anti-HBc, Anti-HBc IgM, Anti-HBe, HBeAg, Anti-HBsAg, HBsAg, HBsAg confirmatory, HBsAg quantitative, Anti-HCV, Chagas 4, CMV IgG, CMV IgG Avidity, CMV IgM, HIV combi PT, HIV-Ag, HIV-Ag confirmatory, HSV-1 IgG, HSV-2 IgG, HTLV-I/II, Rubella IgG, Rubella IgM, Syphilis, Toxo IgG, Toxo IgG Avidity, Toxo IgM, TPLA (Syphilis), Anti-CCP, ASLO, C3c, C4, Ceruloplasmin, CRP (Latex), Haptoglobin, IgA , IgE, IgG, IgM, Immunglobulin A CSF, Immunglobulin M CSF, Interleukin 6, Kappa light chains, Kappa light chains free6, 2,3, Lambda light chains, Lambda light chains free6, 2,3, Prealbumin, Procalcitonin, Rheumatoid factor, a1-Acid Glycoprotein, a1-Antitrypsin, Bicarbonate (CO2), Calcium, Chloride, Fructosamine, Glucose, HbA1c (hemolysate), HbA1c (whole blood), Insulin, Lactate, LDL Cholesterol direct, Magnesium, Potassium, Sodium, Total Protein, Triglycerides, Triglycerides Glycerol blanked, Vitamin D total, Acid phosphatase, AFP, CA 125, CA 15-3, CA 19-9, CA 72-4, Calcitonin, Cyfra 21-1, hCG plus beta, HE4, Kappa light chains free6, 2,3, Lambda light chains free6, 2,3, NSE, proGRP, PSA free, PSA total, SCC, S-100, Thyreoglobulin (TG II), Thyreoglobulin confirmatory, b2-Microglobulin, Albumin (BCG), Albumin (BCP), Albumin immunologic, Creatinine (enzymatic), Creatinine (Jaffe), Cystatin C, Potassium, PTH, PTH (1-84), Total Protein, Total Protein, Urine/CSF, Urea/BUN, Uric acid, a1-Microglobulin, b2-Microglobulin, Acetaminophen (Paracetamol), Amikacin, Carbamazepine, Cyclosporine, Digitoxin, Digoxin, Everolimus, Gentamicin, Lidocaine, Lithium, ISE Mycophenolic acid, NAPA, Phenobarbital, Phenytoin, Primidone, Procainamide, Quinidine, Salicylate, Sirolimus, Tacrolimus, Theophylline, Tobramycin, Valproic Acid, Vancomycin, Anti Muellerian Hormone, AFP, b-Crosslaps, DHEA-S, Estradiol, FSH, free ßhCG, hCG, hCG plus beta, hCG STAT, HE4, LH, N-MID Osteocalcin, PAPP-A, PlGF, sFIt-1, P1NP, Progesterone, Prolactin, SHBG, Testosterone, CMV IgG, CMV IgG Avidity, CMV IgM, Rubella IgG, Rubella IgM, Toxo IgG, Toxo IgG Avidity, and/or Toxo IgM.

As used herein, the terms “complementary” or “complementarity” are used in reference to polynucleotides (i.e., a sequence of nucleotides) related by the customary base-pairing rules. For example, for the sequence “A-G-T”, is complementary to the sequence “T-C-A”. Complementarity may be “partial”, in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands.

As used herein, the term “homology” refers to a degree of complementarity. Homology includes partial homology or complete homology (i.e., identity). A partially complementary sequence, for example, is one that at least partially inhibits a completely complementary sequence from hybridizing to a target nucleic acid and is referred to using the functional term “substantially homologous”. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous to a target under conditions of low stringency. However, conditions of low stringency ca exist and are such that non-specific binding is permitted; low stringency conditions require that the binding of two sequences to one another be a specific (i.e., selective) interaction. The absence of non-specific binding may be tested by the use of a second target that lacks even a partial degree of complementarity (e.g., less than about 30% identity); in the absence of non-specific binding the probe will not hybridize to the second non-complementary target.

The term “ligand” or “analyte ligand” as used herein refers broadly to any compound, molecule, molecular group, or other substance that binds to another entity (e.g., receptor) to form a larger complex. For example, an analyte ligand is an entity that has binding affinity for an analyte, as that term is understood in the art and broadly defined herein. Examples of analyte ligands include, but are not limited to, peptides, carbohydrates, nucleic acids, antibodies, or any molecules that bind to receptors. In certain examples, the ligand forms a complex with an analyte to serve a biological purpose. As those skilled in the art will appreciate, the relationship between a ligand and its binding partner (e.g., an analyte) is a function of charge, hydrophobicity, and/or molecular structure. Binding can occur via a variety of intermolecular forces, such as ionic bonds, hydrogen bonds, and Van der Waals forces. In certain examples, the ligand or analyte ligand is an antibody or functional fragment thereof having binding affinity with an antigen.

As used herein, the term “DNA” refers to a molecule comprising at least one deoxyribonucleotide residue. A “deoxyribonucleotide” is a nucleotide without a hydroxyl group and instead a hydrogen at the 2′ position of a β-D-deoxyribofuranose moiety. The term encompasses double stranded DNA, single stranded DNA, DNAs with both double stranded and single stranded regions, isolated DNA such as partially purified DNA, essentially pure DNA, synthetic DNA, recombinantly produced DNA, as well as altered DNA, or analog DNA, that differs from naturally occurring DNA by the addition, deletion, substitution, and/or modification of one or more nucleotides.

As used herein, the term “join”, “joined”, “link”, “linked”, or “tethered” refers to any method known in the art for functionally connecting two or more entities, such as connecting a protein to a DA molecule or a protein to a protein. For example, one protein may be linked to another protein via a covalent bond, such as in a recombinant fusion protein, with or without intervening sequences or domains. Example covalent linkages may be formed, for example, through SpyCatcher/SpyTag interactions, cysteine-maleimide conjugation, or azide-alkyne click chemistry, as well as other means known in the art. Further, one DNA molecule can be linked to another DNA molecule via hybridization of complementary DNA sequences.

As used herein, the term “nanopore” generally refers to a pore, channel, or passage formed or otherwise provided in a membrane. A membrane may be an organic membrane, such as a lipid bilayer, or a synthetic membrane, such as a membrane formed of a polymeric material. The membrane may be a polymeric material. The nanopore may be disposed adjacent or in proximity to a sensing circuit or an electrode coupled to a sensing circuit, such as, for example, a complementary metal-oxide semiconductor (CMOS) or field effect transistor (FET) circuit. In some example embodiments, a nanopore has a characteristic width or diameter on the order of 0.1 nanometers (nm) to about 1000 nm. Some nanopores are proteins. Alpha-hemolysin monomers, for example, oligomerize to form a protein. The membrane includes a trans side (i.e., side facing the sensing electrode) and a cis side (i.e., side facing the counter electrode).

The term “nucleic acid molecule” or “nucleic acid” includes RNA, DNA and cDNA molecules. It will be understood that, as a result of the degeneracy of the genetic code, a multitude of nucleotide sequences encoding a given protein such as alpha-hemolysin and/or variants thereof may be produced. The present disclosure contemplates every possible variant nucleotide sequence, encoding variant alpha-hemolysin, all of which are possible given the degeneracy of the genetic code.

The term “nucleotide” is used herein as recognized in the art to include natural bases (standard), and modified bases well known in the art. Such bases are generally located at the 1′ position of a nucleotide sugar moiety. Nucleotides generally comprise a base, sugar, and a phosphate group.

As used herein, “synthetic”, such as with reference to, for example, a synthetic nucleic acid molecule or a synthetic gene or a synthetic peptide refers to a nucleic acid molecule or polypeptide molecule that is produced by recombinant methods and/or by chemical synthesis methods.

As used herein, production by recombinant methods by using recombinant DNA methods refers to the use of the well-known methods of molecular biology for expressing proteins encoded by cloned DNA. For example, standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference in its entirety for any purpose.

As used herein, “vector” (or plasmid) refers to discrete DNA elements that are used to introduce heterologous nucleic acid into cells for either expression or replication thereof. The vectors typically remain episomal, but can be designed to effect integration of a gene or portion thereof into a chromosome of the genome. Also contemplated are vectors that are artificial chromosomes, such as bacterial artificial chromosomes, yeast artificial chromosomes and mammalian artificial chromosomes. Selection and use of such vehicles are well known to those of skill in the art.

As used herein, “expression” refers generally to the process by which a nucleic acid is transcribed into mRNA and translated into peptides, polypeptides, or proteins. If the nucleic acid is derived from genomic DNA, expression can, if an appropriate eukaryotic host cell or organism is selected, include processing, such as splicing of the mRNA.

As used herein, an “expression vector” includes vectors capable of expressing DNA that is operatively linked with regulatory sequences, such as promoter regions, that are capable of effecting expression of such DNA fragments. Such additional segments can include promoter and terminator sequences, and optionally can include one or more origins of replication, one or more selectable markers, an enhancer, a polyadenylation signal, and the like. Expression vectors are generally derived from plasmid or viral DNA, or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome. As used herein, vector also includes “virus vectors” or “viral vectors”. Viral vectors are engineered viruses that are operatively linked to exogenous genes to transfer (as vehicles or shuttles) the exogenous genes into cells.

By the term “host cell”, it is meant a cell that contains a vector and supports the replication, and/or transcription or transcription and translation (expression) of the expression construct. Host cells can be prokaryotic cells, such as E. coli or Bacillus subtilus, or eukaryotic cells such as yeast, plant, insect, amphibian, or mammalian cells. In general, host cells are prokaryotic, e.g., E. coli.

The terms “cellular expression” or “cellular gene expression” generally refer to the cellular processes by which a biologically active polypeptide is produced from a DNA sequence and exhibits a biological activity in a cell. As such, gene expression involves the processes of transcription and translation, but can also involve post-transcriptional and post-translational processes that can influence a biological activity of a gene or gene product. These processes include, for example, RNA synthesis, processing, and transport, as well as polypeptide synthesis, transport, and post-translational modification of polypeptides. Additionally, processes that affect protein-protein interactions within the cell can also affect gene expression as defined herein.

As used herein, the term “optional” or “optionally” means that the subsequently described event or circumstance does or does not occur, and that the description includes instances where said event or circumstance occurs and instances where it does not. For example, an optional step of joining an analyte detection complex to a nanopore assembly monomer means that that the analyte detection complex can be joined or not joined.

The term “phospholipid” as used herein, refers to a hydrophobic molecule comprising at least one phosphorus group. For example, a phospholipid can comprise a phosphorus-containing group and saturated or unsaturated alkyl group, optionally substituted with OH, COOH, oxo, amine, or substituted or unsubstituted aryl groups.

As used herein, the term “membrane” refers to a sheet or layer of continuous double layer of lipid molecules, in which membrane proteins are embedded. Membrane lipid molecules are typically amphipathic, and most spontaneously form bilayers when placed in water. A “phospholipid membrane” refers to any structure composed of phospholipids aligned such that the hydrophobic heads of the lipids point one way while the hydrophilic tails point the opposite way. Examples of phospholipid membranes include the lipid bilayer of a cellular membrane.

As used herein, “identity” or “sequence identity” refers to, in the context of a sequence, the similarity between two nucleic acid sequences, or two amino acid sequences, and is expressed in terms of the similarity between the sequences, otherwise referred to as sequence identity. Sequence identity is frequently measured in terms of percentage identity (or similarity or homology); the higher the percentage, the more similar the two sequences are. For example, 80% homology means the same thing as 80% sequence identity determined by a defined algorithm, and accordingly a homologue of a given sequence has greater than 80% sequence identity over a length of the given sequence. Example levels of sequence identity include, for example, 80, 85, 90, 95, 98% or more sequence identity to a given sequence, e.g., the coding sequence for any one of the inventive polypeptides, as described herein.

Methods of alignment of sequences for comparison are well known in the art. Various programs and alignment algorithms are described in: Smith & Waterman Adv. Appl. Math. 2: 482, 1981; Needleman & Wunsch J. Mol. Biol. 48: 443, 1970; Pearson & Lipman Proc. Natl. Acad. Sci. USA 85: 2444, 1988; Higgins & Sharp Gene 73: 237-244, 1988; Higgins & Sharp CABIOS 5: 151-153, 1989; Corpet et al. Nuc. Acids Res. 16, 10881-90, 1988; Huang et al. Computer Appls. In the Biosciences 8, 155-65, 1992; and Pearson et al. Meth. Mol. Bio. 24, 307-31, 1994. Altschul et al. (J. Mol. Biol. 215:403-410, 1990), presents a detailed consideration of sequence alignment methods and homology calculations.

The NCBI Basic Local Alignment Search Tool (BLAST) (Altschul et al. J. Mol. Biol. 215:403-410, 1990) is available from several sources, including the National Center for Biotechnology Information (NCBI, Bethesda, Md.) and on the Internet, for use in connection with the sequence analysis programs that include, for example, the suite of BLAST programs, such as BLASTN, BLASTX, and TBLASTX, BLASTP and TBLASTN.

Sequence searches are typically carried out using the BLASTN program when evaluating a given nucleic acid sequence relative to nucleic acid sequences in the GenBank DNA Sequences and other public databases. The BLASTX program is preferred for searching nucleic acid sequences that have been translated in all reading frames against amino acid sequences in the GenBank Protein Sequences and other public databases. Both BLASTN and BLASTX are run using default parameters of an open gap penalty of 11.0, and an extended gap penalty of 1.0, and utilize the BLOSUM-62 matrix. (See, e.g., Altschul, S. F., et al., Nucleic Acids Res. 25:3389-3402, 1997).

In certain example embodiments, a preferred alignment of selected sequences in order to determine “% identity” between two or more sequences, is performed using for example, the CLUSTAL-W program in MacVector version 13.0.7, operated with default parameters, including an open gap penalty of 10.0, an extended gap penalty of 0.1, and a BLOSUM 30 similarity matrix.

As used herein, the term “variant” refers to a modified protein which displays altered characteristics when compared to the parental protein, e.g., altered ionic conductance.

As used herein, the term “sample” or “test sample” is used in its broadest sense. A “biological sample”, as used herein, includes, but is not limited to, any quantity of a substance from a living thing or formerly living thing, such as from a subject. A biological sample can include a sample of biological tissue or fluid origin obtained in vivo or in vitro. Such samples can be from, without limitation, body fluids, organs, tissues, fractions, and cells isolated from a biological subject. Biological samples can also include extracts from a biological sample, such as for example an extract from a biological fluid (e.g., blood or urine).

As used herein, a “biological fluid” or “biological fluid sample” refers to any physiologic fluid (e.g., blood, blood plasma, sputum, lavage fluid, ocular lens fluid, cerebrospinal fluid, urine, semen, sweat, tears, milk, saliva, synovial fluid, peritonaeal fluid, amniotic fluid), as well as solid tissues that have, at least in part, been converted to a fluid form through one or more known protocols or for which a fluid has been extracted. For example, a liquid tissue extract, such as from a biopsy, can be a biological fluid sample. In certain examples, a biological fluid sample is a urine sample collected from a subject. In certain examples, the biological fluid sample is a blood sample collected from a subject. As used herein, the terms “blood”, “plasma” and “serum” include fractions or processed portions thereof. Similarly, where a sample is taken from a biopsy, swab, smear, etc., the “sample” encompasses a processed fraction or portion derived from the biopsy, swab, smear, etc.

Further, a “fluid solution”, “fluid sample” or “fluid” encompass biological fluids but can also include and encompass non-physiological components, such as any analyte that may be present in an environmental sample. For example, the sample may be from a river, lake, pond, or other water reservoir. In certain example embodiments, the fluid sample can be modified. For example, a buffer or preservative can be added to the fluid sample, or the fluid sample can be diluted. In other example embodiments, the fluid sample can be modified by common means known in the art to increase the concentration of one or more solutes in the solution. Regardless, the fluid solution is still a fluid solution as described herein. When a fluid sample is to be tested, for example, the fluid sample can be referred to as a “test sample”.

As used herein, a “subject” refers to an animal, including a vertebrate animal. The vertebrate can be a mammal, for example, a human. In certain examples, the subject can be a human patient. A subject can be a “patient”, for example, such as a patient suffering from or suspected of suffering from a disease or condition and can be in need of treatment or diagnosis or can be in need of monitoring for the progression of the disease or condition. The patient can also be in on a treatment therapy that needs to be monitored for efficacy. A mammal refers to any animal classified as a mammal, including, for example, humans, chimpanzees, domestic and farm animals, as well as zoo, sports, or pet animals, such as dogs, cats, cattle, rabbits, horses, sheep, pigs, and so on.

As used herein, the term “wild-type” refers to a gene or gene product which has the characteristics of that gene or gene product when isolated from a naturally-occurring source.

The following examples and figures are provided to aid the understanding of the present invention, the true scope of which is set forth in the appended claims. It is understood that modifications can be made in the procedures set forth without departing from the spirit of the invention.

### DESCRIPTION OF THE FIGURES

FIG. 1 is an illustration showing an analyte detection complex, in accordance with certain example embodiments.

FIG. 2A is an illustration showing three nanopore assemblies, each including an analyte detection complex directed to a different analyte, in accordance with certain example embodiments.

FIG. 2B is an illustration showing the three nanopore assemblies of FIG. 2A, but with each of the analyte ligands shown binding their respective analytes, in accordance with certain example embodiments.

FIG. 2C is an illustration showing the same three nanopore assemblies as in FIGS. 2A-2B, except that the nanopore assemblies are shown in a configuration in which each analyte detection complex is being pulled towards the trans side of the nanopore assembly, in accordance with certain example embodiments.

FIG. 3 is an illustration showing assessment of a weak binding interaction between an analyte ligand and an analyte, along with the electrical signal changes associated with the binding and dissociation of the analyte-ligand pair, in accordance with certain example embodiments.

FIG. 4 is an illustration showing assessment of a strong binding interaction between an analyte ligand and an analyte, in accordance with certain example embodiments.

FIG. 5 is an illustration showing assessment of a very strong interaction between an analyte ligand and an analyte, in accordance with certain example embodiments.

FIG. 6 is an illustration showing assessment of a test sample when the target analyte is absent from a test solution, in accordance with certain example embodiments.

FIG. 7 is an illustration showing an example confidence level distribution of individual analyte captures and dissociations for weak, strong, and very strong analyte-ligand interactions, in accordance with certain example embodiments.

FIG. 8 is an illustration showing the identification of specific analyte-ligand interactions on a chip, in accordance with certain example embodiments.

### EXAMPLE EMBODIMENTS

The example embodiments are now described in detail, in part with reference to the accompanying figures. Where figures are referenced, like numerals indicate like (but not necessarily identical) elements throughout the figures.

### Analyte Detection Complex

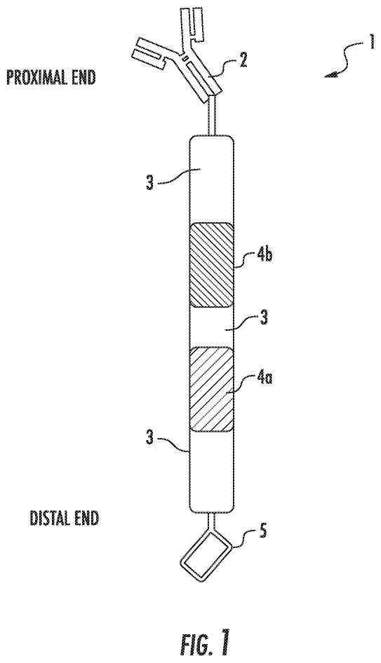

FIG. 1 is an illustration of an analyte detection complex 1, in accordance with certain example embodiments. With reference to FIG. 1, the analyte detection complex 1 includes, for example, an analyte ligand 2, a threading element 3, and one or more signal elements 4a and 4b that are disposed within or associated with the threading element 3. In certain example embodiments, the analyte detection complex 1 also includes an anchoring tag 5 that is located on the distal end of the analyte detection complex.

The analyte ligand 2 of the analyte detection complex 1 can be any ligand that has binding affinity to any analyte as described herein. As shown in FIG. 1, for example, the analyte ligand 2 can be an antibody with the analyte being an antigen having binding affinity for the antibody. As those skilled in the art will appreciate in view of this disclosure, any antibody or functional fragment thereof can be used as the analyte ligand. In other example embodiments, the analyte ligand 2 of the analyte detection complex 1 can be used to detect an environmental analyte. In certain example embodiments, the analyte ligand 2 of the analyte detection complex 1 can be used to identify protein analytes in complex biological fluid samples, for example, in a tissue and/or a bodily fluid.

In certain example embodiments, the analyte to which the analyte ligand 2 is directed can be present in a low concentration as compared to other components of the biological or environmental sample. In certain examples embodiments, the analyte ligand 2 can also be used to target subpopulations of macromolecular analytes based on conformation or on functional properties of the analytes. Example analyte ligands 2 include those defined herein as well as aptamers, antibodies or functional fragments thereof, receptors, and/or peptides that are known to bind to the target analyte. With regard to aptamers, the aptamer can be a nucleic acid aptamer including DNA, RNA, and/or nucleic acid analogs. In certain example embodiments, the aptamer may be a peptide aptamer, such as a peptide aptamer that includes a variable peptide loop attached at both ends to a scaffold. Aptamers can be selected, for example, to bind to a specific target protein analyte.

As those skilled in the art will appreciate, an analyte and analyte ligand 2 represent two members of a binding pair, i.e., two different molecules in which one of the molecules specifically binds to the second molecule through chemical and/or physical interactions. In addition to the well-known antigen-antibody binding pair members, other binding pairs include, for example, biotin and avidin, carbohydrates and lectins, complementary nucleotide sequences, complementary peptide sequences, effector and receptor molecules, enzymes cofactors and enzymes, enzyme inhibitors and enzymes, a peptide sequence and an antibody specific for the sequence or the entire protein, polymeric acids and bases, dyes and protein binders, peptides and specific protein binders (e.g., ribonuclease, S-peptide and ribonuclease S-protein), sugar and boronic acid, and similar molecules having an affinity which permit their associations in a binding assay.

Further, analyte-ligand binding pairs can include members that are analogs of the original binding member, e.g., an analyte-analog or binding member made by recombinant techniques or molecular engineering. If the analyte ligand is an immunoreactant it can be, e.g., an antibody, antigen, hapten, or complex thereof, and if an antibody is used, it can be a monoclonal or polyclonal antibody, a recombinant protein or antibody, a chimeric antibody, a mixture(s) or fragment(s) thereof, as well as a mixture of an antibody and other binding members. The details of the preparations of such antibodies, peptides and nucleotides and their suitability for use as binding members in a binding assay are well known in the art.

As shown in FIG. 1, the analyte ligand 2, such as an antibody, is joined to a threading element 3. When associated with a nanopore, the threading element 3 can thread through the pore of a nanopore. The threading element 3 can be any structure that can thread through the pore of a nanopore assembly. In certain example embodiments, the threading element 3 can be a single or double stranded nucleic acid sequence or other molecular polymer. For example, the threading element 3 can be an amino acid sequence and can include carbon spacers. In certain example embodiments, the threading element 3 has an overall charge of one polarity, and the changing the voltage across a nanopore assembly as described herein can cause the threading element to move in one direction or another.

Associated with the threading element 3 of the analyte detection complex 1 are one or more signal elements, such as 1, 2, 3, 4 or 5 signal elements. As shown in FIG. 1, for example, the threading element 3 can be associated with a pair of signal elements 4a and 4b. When positioned in the pore of a nanopore, the one or more signal elements 4a and 4b, for example, can be used to determine the location of the threading element 3 within the nanopore assembly. The signal element, for example, can be used to provide an optical, electrochemical, magnetic, or electrostatic (e.g., inductive, capacitive) signal, the signal being detectable and providing an indication of the location of the threading element 3 within the pore of a nanopore assembly as described herein. In certain example embodiments, the signal element 4a can be the same as the signal element 4b. In other example embodiments, the single element 4a can be different than signal element 4b. In certain example embodiments, when the overall charge of the threading element 3 is a given charge, the signal element can represent constriction site of specific charge that can be used to determine the location of the threading element in the pore a nanopore assembly.

In certain example embodiments, the signal element can be an oligonucleotide, a peptide, or polymer sequence that is associated with threading element 3. In certain example embodiments, the signal element can be integrated as part of the threading element 3, such as when the threading element 3 is a nucleotide sequence and the signal element is a specific sequence within the nucleotide sequence of the threading element 3. For example, the signal element can be a subsection of the threading element. Additionally or alternatively, the signal element can be attached to the threading element 3.

The one or more signal elements, such as signal elements 4a and 4b, can be associated with a variety of locations on the threading element 3 so that, when in use, a variety of different signals and/or signal changes can be detected as described herein. For example, when signal element 4a and 4b are different, the electrical signal associated with a nanopore assembly can be different depending on which signal element—4a or 4b—is located within the pore, as described herein. In certain example embodiments, the one or more signal elements can be located on the proximal end of the threading element, while in other example embodiments the one or more signal elements 4 can be located more distally on the analyte detection complex 1. In other example embodiments, one signal element 4a can be associated with the proximal end of the threading element 3, while another signal element 4b can be associated the more distal portion of the threading element 3.

In certain example embodiments, the one or more signal elements, such signal elements 4a and 4b, can be a single stranded nucleic acid sequence, such as a series of repeated nucleic acid residues. For example, the signal element can be a repeated, single-stranded oligonucleotide sequence about 10-100 nucleotides in length, such as about 5, 10, 15, 20, 25, 30, 35, 40, 45, 50, 55, 60, 65, 70, 75, 80, 85, 90, 95, or 100 nucleotides. In certain example embodiments, the signal element can be a 30-50 oligonucleotide sequence, such as a 40 mer oligonucleotide sequence.

In other example embodiments, the one or more signal elements can be a double stranded nucleic acid sequence, such as a series of repeated nucleic acid base pairs. For example, the signal element can be a repeated, double stranded oligonucleotide sequence about 10-100 nucleotides in length, such as about 10, 15, 20, 25, 30, 35, 40, 45, 50, 55, 60, 65, 70, 75, 80, 85, 90, 95, or 100 base pairs. In certain example embodiments, the signal element can be a 30-50 oligonucleotide sequence, such as a 40 mer base-pair sequence. In certain example embodiments, the one or more signal elements can include a series of T residues and a series of N3-cyanoethyl-T residues. In certain example embodiments, the signal element of the threading element can include Sp2 units, Sp3 units, dSp units, methylphosphonate-T units, etc.

As shown in FIG. 1, the analyte detection complex 1 also includes an anchoring tag 5 on the distal end of the analyte detection complex 1. When the analyte detection complex 1 is threaded through a nanopore, for example, the anchoring tag 5 can be used to prevent the analyte detection complex 1 from migrating through or, as described herein, being pulled through to the cis side of the nanopore assembly. Hence, the anchoring tag 5 can be any protein, nucleic acid, or chemical entity that can be used to anchor the distal end of the analyte detection complex 1 to the trans side of a nanopore assembly. For example, the anchoring tag 5 can be biotin-streptavidin, double stranded DNA or RNA, DNA or RNA ternary structures, SpyTag-Catcher, antibody-antigen.

### Nanopore Assemblies

In certain example embodiments, the analyte detection complex 1 described herein is associated with a nanopore to form a nanopore assembly and used therewith to interact with an analyte. To detect the interaction of an analyte detection complex 1 with an analyte, the nanopore assembly including the analyte detection complex 1 is embedded within a membrane, and a sensing electrode is positioned adjacent to or in proximity to the membrane. For example, the nanopore assembly including the analyte detection complex 1 can be formed or otherwise embedded in a membrane disposed adjacent to a sensing electrode of a sensing circuit, such as an integrated circuit. The integrated circuit can be an application specific integrated circuit (ASIC). In certain example embodiments, the integrated circuit is a field effect transistor or a complementary metal-oxide semiconductor (CMOS). The sensing circuit can be situated in a chip or other device including the nanopore, or off of the chip or device, such as in an off-chip configuration. The semiconductor can be any semiconductor, including, without limitation, Group IV (e.g., silicon) and Group III-V semiconductors (e.g., gallium arsenide). See, for example, WO 2013/123450, for the apparatus and device set-up that can be used in accordance with the compositions and methods described herein, the entire contents of which are hereby expressly incorporated herein by reference.

As those skilled in the art will appreciate, pore based sensors (e.g., biochips) can be used for electro-interrogation analysis of single molecules. A pore based sensor can include a nanopore assembly as described herein that is formed in a membrane that is disposed adjacent or in proximity to a sensing electrode. The sensor can include, for example, a counter electrode. The membrane includes a trans side (i.e., side facing the sensing electrode) and a cis side (i.e., side facing the counter electrode). Hence, a nanopore assembly that is disposed in the membrane also includes a trans side (i.e., side facing the sensing electrode) and a cis side (i.e., side facing the counter electrode). As described herein, for example, the analyte ligand 2 is located on the cis side of the nanopore assembly, while the anchoring tag 5 is located on the trans side of the nanopore assembly.

The nanopore of the nanopore assembly is typically a multimeric protein embedded in a substrate, such as a membrane. Examples of protein nanopores include, for example, alpha-homolysin, voltage-dependent mitochondrial porin (VDAC), OmpF, OmpC, OmpG, MspA and LamB (maltoporin) (see Rhee, M. et al., Trends in Biotechnology, 25(4) (2007): 174-181). Other example nanopores include phi 29 DNA-packaging nanomotor, ClyA, FhuA, aerolysin, and Sp1. In certain example embodiments, the nanopore protein can be a modified protein, such as a modified natural protein or synthetic protein. In the case of alpha-hemolysin, for example, the nanopore of the nanopore assembly can be an oligomer of seven alpha-hemolysin monomers (i.e., a heptameric nanopore assembly). The monomeric subunits of the alpha-hemolysin heptameric nanopore assembly can be identical copies of the same polypeptide or they can be different polypeptides, so long as the ratio totals seven subunits. The nanopore can be assembled by any method known in the art. For example, an alpha-hemolysin nanopore assembly can be assembled according to the methods described in WO2014/074727, which is hereby incorporated herein in its entirety.

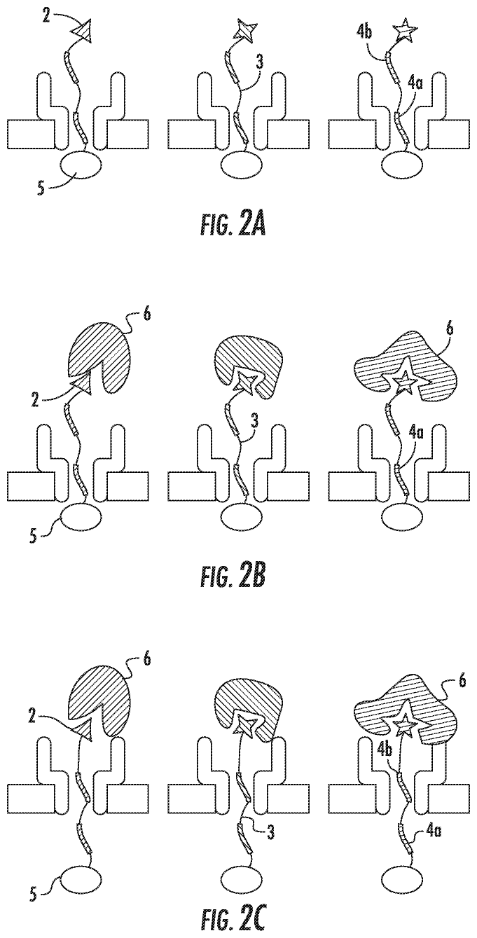

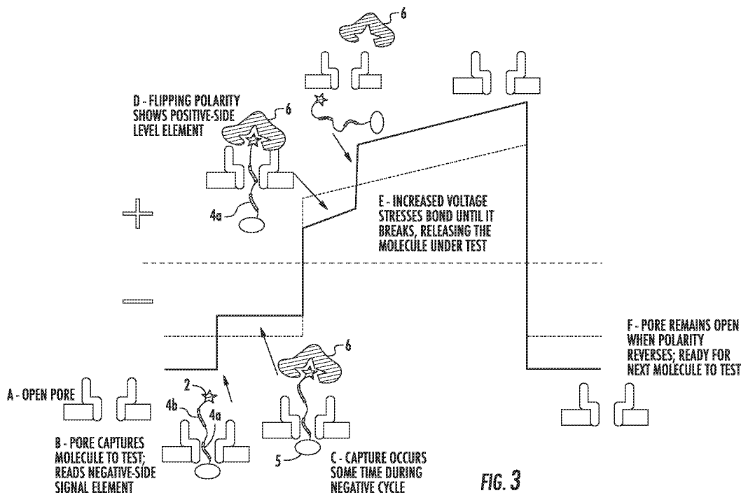

With reference to FIG. 2A, provided is an illustration showing three nanopore assemblies, each of which include an analyte detection complex 1, in accordance with certain example embodiments. As shown, the proximal end of the analyte detection complex 1, including the analyte ligand 2, is located on the cis side of the nanopore assembly. As such, the analyte ligand 2 of the analyte detection complex 1 can be presented to analytes on the cis side of the nanopore assembly, thereby facilitating binding of the analyte ligand 2 to the analyte as described herein. In the example shown in FIG. 2, each analyte ligand 2 is directed to a different analyte ligand. Further, the anchoring tag 5 is located on the trans side of the nanopore assembly (FIG. 2A). The threading element 3, for example, extends through the pore of the nanopore, thereby positioning one or more of the signal elements (e.g., 4a or 4b) within the pore of the nanopore assembly. As shown, a first signaling element 4a is located within the pore of the nanopore assembly, while a second signaling element 4b is located on the cis side of the pore. Each nanopore assembly, for example, can be disposed within an individual well of the biochip.

With reference to FIG. 2B, provided is an illustration showing the three nanopore assemblies of FIG. 2A, but with each of the analyte ligands 2 shown binding their respective analytes 6, in accordance with certain example embodiments. The nanopore assemblies are also shown in a configuration in which the analyte detection complexes are being pulled towards the cis side of the nanopore assembly. Like FIG. 2A, each analyte ligand 2 is located on the cis side of the nanopore assembly, and hence analyte binding occurs on the cis side of the nanopore assembly (FIG. 2B). And as with FIG. 2A, the first signal element 4a of the threading element 3 remains located within the pore of the nanopore assembly, while the second signaling element 4b of the threading element 3 is located on the cis side of the nanopore assembly (FIG. 2B).

With reference to FIG. 2C, provided is an illustration showing the same three nanopore assemblies as in FIGS. 2A-2B, except that the nanopore assemblies are shown in a configuration in which each analyte detection complex is being pulled towards the trans side of the nanopore assembly, in accordance with certain example embodiments. As shown, the second signal 4b of the threading element 3 is now located within the pore of the nanopore assembly, while the first signal element 4a of the threading element 3 has moved to the trans side of the nanopore assembly. In the examples shown in FIG. 2C, the binding of the analytes to their respective analyte ligands can prevent the analyte detection complexes from moving to the trans side of the nanopore assemblies. As described further below, however, if the force pulling the analyte detection complex to the trans side of the nanopore assembly overcomes the bonding force of the analyte-ligand interaction, the analyte ligand 3 of the analyte detection complex 1 can dissociate from the analyte. The analyte detection complex 1 can then translocate to the trans side of the nanopore assembly.

### Methods & Systems for Assessing Analyte-Ligand Interactions

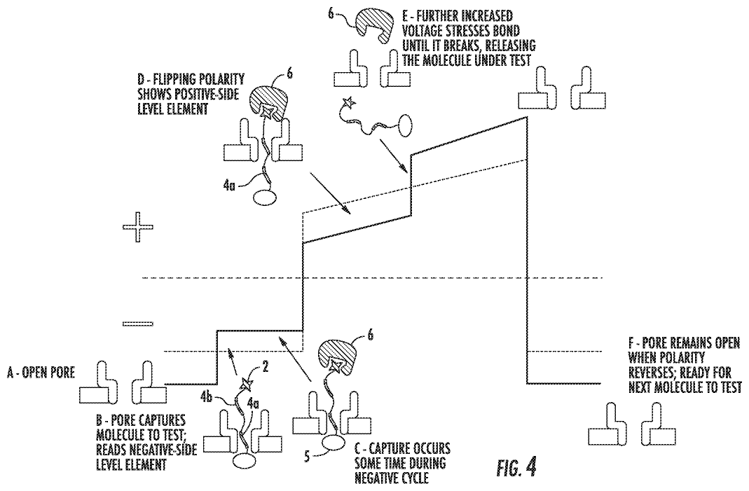

In certain example embodiments, provided are methods and systems for assessing binding interactions between a ligand and the ligand's analyte, including assessing binding strength between the analyte ligand and the analyte. For example, a nanopore assembly including an analyte detection complex 1 as described herein can be incorporated into a biochip. The biochip can then be contacted with a fluid sample that is to be analyzed. If the analyte is present in the fluid solution, the analyte ligand 2 of the analyte detection complex 1 can bind the analyte, thereby resulting in a discernable electrical signal associated with the nanopore assembly (i.e., a binding signal). Further, the binding strength between the analyte ligand 2 and the analyte can be determined based on the electrical s associated with the pore. If the analyte is not present in the fluid sample, then the analyte ligand 2 does not bind the analyte, in which case the absence of a binding event can be determined from the electrical signals associated with the nanopore assembly. Without wishing to be bound by any particular theory, such methods and systems are illustrated in FIGS. 3-8.

With reference to FIG. 3, provided is an illustration showing assessment of a weak binding interaction between an analyte ligand 2 and an analyte 6, along with the electrical signal changes associated with the binding and dissociation of the analyte-ligand pair, in accordance with certain example embodiments. As shown at point “A” of FIG. 3, a nanopore can be disposed within a membrane of a chip as an “open pore”. That is, in certain example embodiments, the pore may not initially include an analyte detection complex 1, in which case a baseline electrical signal can be obtained from the nanopore via the electrodes associated with the pore. As a first voltage is applied across the nanopore assembly, for example, in certain example embodiments the nanopore can capture an analyte detection complex 1, thereby locating a first signal element 4a within the within the pore (see point “B”) and forming a nanopore assembly, as described herein.

In certain example embodiments, an electrical signal can be detected from the nanopore assembly at point “B”, the signal indicating the threading of the analyte detection complex 1 within the nanopore of the nanopore assembly (FIG. 3). For example, the signal can be a threading signal that corresponds to the presence of the first signal element 4a being positioning in the pore of the nanopore (FIG. 3). As show in FIG. 3, for example, application of the first voltage also pulls the analyte detection complex 1 towards the cis side of the nanopore assembly. The anchoring tag 5, however, can prevent the analyte detection complex 1 from being pulled through to the cis side of the membrane. For example, the size of the anchoring tag 5 relative to the size of the pore can prevent the analyte detection complex 1 from translocating to the cis side of the nanopore assembly.

Once the analyte detection complex 1 is located within the nanopore, for example, the chip—and hence the nanopore assembly disposed within the chip membrane—is contacted with a fluid sample. That is, the nanopore assembly is contacted with a sample that is to be tested or examined, such as for the presence of the target analyte 6. For example, to test a fluid solution for the presence of the analyte, the fluid solution can be flowed over a nanopore assembly that is arranged to include an analyte detection complex 1 as described herein, with the analyte ligand 2 of the analyte detection complex 1 having binding affinity to the target analyte.

As the fluid is flowed over the nanopore assembly, the analyte 6 (when present) has an opportunity to contact the analyte ligand 2 of the analyte detection complex 1 and hence can bind the analyte ligand 2. But if the analyte is absent from the fluid solution, no biding of the analyte to the analyte ligand 2 of the analyte detection complex 1 occurs. As shown in the example of FIG. 3, binding of the analyte 6 to the analyte ligand 2 occurs at point “C”. Yet because the analyte 6 is not blocking the pore of the nanopore assembly, for example, the electrical signal associated with the nanopore assembly can remain roughly unchanged. For example, the first signal element 4a can remain positioned in the pore if the nanopore assembly.

After contacting the chip with the fluid sample, and hence providing an opportunity for any analyte to bind the analyte ligand 2, a second voltage that is opposite in polarity to the first voltage is incrementally applied across the membrane. That is, the first voltage is progressively transitioned to a second voltage that is opposite in polarity to the first voltage. For example, the first voltage may have a negative potential that is then transitioned to a voltage with a positive potential. As shown in FIG. 3, for example, positioning the analyte 6 detection complex 1 in an open pore and binding of an analyte ligand 2 to an analyte may occur in a negative cycle, with the voltage thereafter being slowly changed to a second (positive) voltage that is opposite in polarity to the first voltage.

As the voltage opposite in polarity to the first voltage is incrementally applied across the membrane, for example, the analyte ligand 2 and its bound analyte 6 are pulled towards the trans side of the nanopore assembly (point “D” of FIG. 3). The bound analyte 6, however, can prevent the analyte detection complex 1 from pulling through the nanopore assembly to the trans side of the nanopore assembly. Further, the second signal element 4b (e.g., a positive side signal element) can be positioned within the pore of the nanopore assembly.

As shown in FIG. 3, binding of the analyte 6 to the analyte ligand 2 and repositioning of the analyte detection complex 1 within the pore can result in a binding signal that is different and distinguishable from the threading signal. The binding signal, for example, is a detectable electrical signal associated with the nanopore assembly that corresponds to the presence of the analyte 6 being bound to the analyte ligand 2 (point “D” of FIG. 3). Hence, the detection of the binding signal can also provide an indication that the analyte in present in the tested sample. In certain example embodiments, comparing the threading signal to the binding signal provides the indication that the analyte 6 is bound to the analyte ligand 2 (and hence that the analyte is present in the test sample). For example, the change in electrical signal from the threading signal to a binding signal indicates that the analyte 6 is bound to the analyte ligand 2.

In certain example embodiments, the positioning of the second signal element 4b in the pore of the nanopore assembly results in the binding signal. For example, the second signal element 4b can produce a particular electrical signal that is associated with the second signal element 4b being placed within the nanopore. As such, detection of the electrical signal associated with the second signal element 4b corresponds to the binding signal. Additionally or alternatively, in certain example embodiments the analyte 6 that is bound to the analyte ligand 2 may result in a detectable signal change, such as compared to the threading signal, thereby indicating the presence of the analyte in the sample. For example, and without being bound by any particular theory, the presence of the analyte 6 at or near the pore opening may block or partially block the pore of the nanopore assembly, thereby affecting the electrical signal arising from the nanopore assembly (and resulting in a detectable binding signal).

Following determination of a binding signal, in certain example embodiments the voltage opposite in polarity to the first voltage can be further increased, thereby further increasing the force pulling the analyte detection complex 1 towards the trans side of the nanopore assembly. At some point while the voltage is increased, the force pulling the analyte detection complex 1 towards the trans side of the nanopore assembly can become strong enough to pull the analyte ligand 2 away from the analyte 6. At this point, which is illustrated as point “E” in FIG. 3, the analyte ligand 2 and the analyte 6 can dissociate, and the analyte detection complex 1 moves to the trans side of the nanopore assembly. Hence, any signal element located within the pore can move out of the pore entirely, and the nanopore assembly transitions to an open nanopore state. Further, an electrical signal can be obtained by the electrode associated with the nanopore, the electrical signal corresponding to a dissociation signal. In other words, the dissociation signal corresponds to the electrical signal obtained from the nanopore assembly at or about the time that the analyte ligand 2 dissociates from the analyte 6. As shown in FIG. 3, the interaction between the analyte and the analyte ligand 2 is a weak interaction, as the analyte dissociates from the analyte ligand 2 relatively early as the voltage is increased as described herein.

Once the analyte ligand 2 of the analyte detection complex 1 dissociates from the analyte 6 and the analyte detection complex 1 moves to the trans side of the nanopore, in certain example embodiments the voltage can again be reversed and the pore can be recycled (point “F” of FIG. 3). That is, following the dissociation event described herein, a voltage opposite in polarity to the second voltage can be applied across the membrane. For example, the voltage can be the same or similar in magnitude and polarity to the first voltage described herein. Hence, the pore can then capture an analyte detection complex 1 as described herein for points “A” and “B” of FIG. 3. Thereafter, the process of points “C” through “F” can be repeated. In certain example embodiments, a given nanopore assembly including an analyte detection complex 1 can be reused multiple times during an analysis of a given sample.

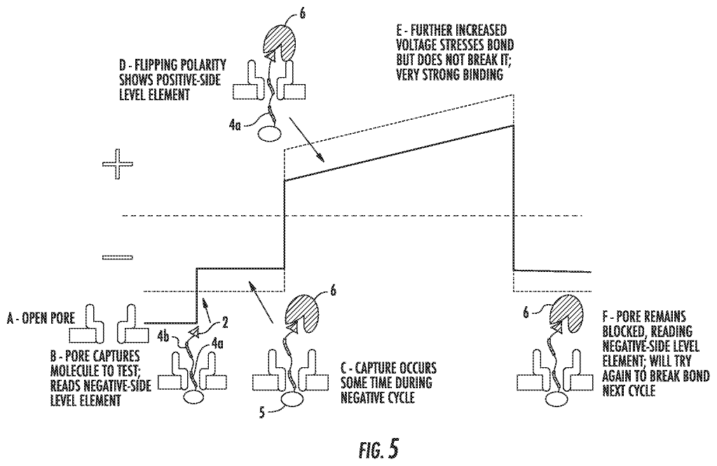

With reference to FIG. 4, provided is an illustration showing assessment of a strong binding interaction between an analyte ligand 2 and an analyte 6, in accordance with certain example embodiments. As shown at point “A” of FIG. 4, a nanopore can be disposed within a membrane of a chip as an “open pore”. As a first voltage is applied across the nanopore assembly, for example—and like the example shown in FIG. 3—in certain example embodiments the nanopore can capture an analyte detection complex 1, thereby locating a first signal element 4a within the within the pore (see point “B”). A threading signal can then be detected from the nanopore assembly at point “B”, the threading signal indicating the presence of the analyte detection complex 1 within the nanopore of the nanopore assembly (FIG. 4). For example, the signal can correspond to the presence of a first signal element 4a being positioning in the pore of the nanopore assembly (FIG. 4). Further, like FIG. 3, the anchoring tag 5 can prevent the analyte detection complex 1 from being pulled to the cis side of the nanopore assembly (FIG. 4).

Once the analyte detection complex 1 is located within the nanopore, for example, the chip is contacted with a fluid sample as described herein, thereby facilitating binding of the analyte ligand 2 to its cognate analyte 6. As shown in FIG. 4, binding of the analyte to the analyte ligand 2 occurs at point “C”. Yet because the analyte 6 is not blocking the pore of the nanopore assembly, for example, the electrical signal associated with the nanopore assembly can remain roughly unchanged (FIG. 4). For example, the first signal element 4a can remain positioned in the pore if the nanopore assembly, while a second signal element 4b can remain on the trans side of the nanopore assembly.

After contacting the chip with the fluid sample, and hence providing an opportunity for an analyte to bind the analyte ligand 2, the second voltage that is opposite in polarity to the first voltage can be incrementally applied across the nanopore assembly. For example, the second voltage is progressively applied across the nanopore assembly. As with the weak binding example of FIG. 2, for example, positioning the analyte detection complex 1 in an open pore and binding of an analyte ligand 2 to an analyte may occur in a negative cycle, with the voltage thereafter being slowly changed to a second (positive) voltage that is opposite in polarity to the first voltage (FIG. 4).

As the voltage opposite in polarity to the first voltage is incrementally applied across the membrane, the analyte ligand 2 and its bound analyte are pulled towards the trans side of the nanopore assembly (point “D” of FIG. 4), as described herein. Further, the second signal element 4b (e.g., a positive side signal element) can be positioned within and remain within the pore of the nanopore, thereby providing a binding signal. Hence, as with the example weak binding example illustrated in FIG. 3, the detection of the binding signal provides an indication that the analyte in present in the tested sample (see point “D” of FIG. 4). And in certain example embodiments, the presence of the bound analyte can additionally or alternatively provide a binding signal, as described herein.

As shown at point “E” of FIG. 4, further increasing the second voltage can result in dissociation of the analyte ligand 2 from the analyte, the dissociation being associated with a discernable dissociation signal. As compared to point “E” in FIG. 3, however, the stronger binding illustrated in FIG. 4 results in more force being required to separate the analyte ligand 2 from the analyte. Hence, as illustrated in FIG. 4, the analyte stays bound to the analyte ligand 2 for a longer period of time (as compared to the weak binding shown in FIG. 3). As such, the dissociation signal associated with the nanopore assembly shown in FIG. 4 (strong binding at point “E”) is different than the dissociation signal shown in FIG. 3 (weak binding at point “E”). Following dissociation of the analyte ligand 2 from the analyte, the analyte detection complex 1 can move to the trans side of the membrane, and the nanopore can be recycled (point “F”, FIG. 4) as described herein.

With reference to FIG. 5, provided is an illustration showing assessment of a very strong interaction between an analyte ligand 2 and an analyte 6, in accordance with certain example embodiments. As shown in FIG. 5, the nanopore assembly progresses through points A-D as described with reference to FIGS. 3 and 4. For example, an analyte 6 binds the analyte ligand 2 at point “C”, and with an incrementally increased application of a second voltage opposite in polarity to the applied first voltage, the analyte detection complex 1 is pulled towards the trans side of the nanopore at point “D”. At point “D”, for example, a dissociation signal can be obtained.

But unlike the analyte-ligand interactions described with reference 2 FIGS. 3 and 4, the binding between the analyte 6 and the analyte ligand 2 is so strong that increasing the second voltage cannot overcome the binding forces between the analyte and the analyte ligand 2 (FIG. 5 at point “E”). Hence, no dissociation signal is obtained, as there is no dissociation between the analyte and the analyte ligand 2 (FIG. 5). As such, the signaling element 4b can remain in the pore throughout the positive-side cycle (with signal element 4a out of the pore, Point “D”), thereby providing an indication that the analyte is very strongly bound to the analyte ligand 2 (FIG. 5). In other words, determination of a binding signal as described herein—followed by the absence of a dissociation signal as described herein—can provide an indication that the analyte has remained bound to the analyte ligand 2 despite the increased second voltage. In such example embodiments, the nanopore is not recycled. As shown in FIG. 5, for example, the analyte remains bound to the analyte ligand 2 even after the voltage opposite in polarity to the second voltage is applied across the nanopore assembly (FIG. 5 at point “F”).

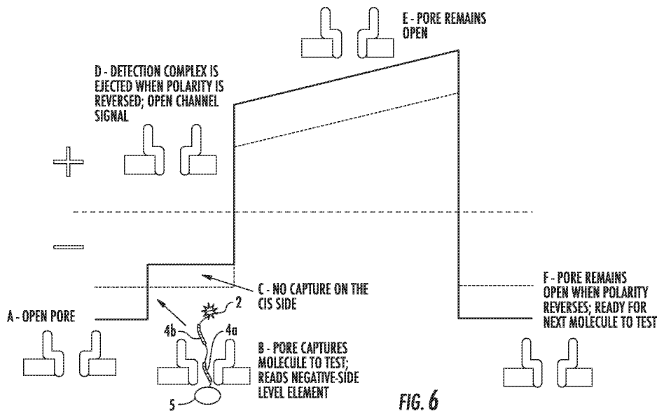

With reference to FIG. 6, provided is an illustration showing assessment of a test sample when the target analyte is absent from a test solution, in accordance with certain example embodiments. As shown in FIG. 6, the nanopore assembly progresses through points A-B as described with reference to FIGS. 3-5. For example, the analyte detection complex 1 can be positioned within the pore of the nanopore assembly at point “B” via application of the first voltage as described herein and a threading signal detected (FIG. 6). As shown signal element 4a locates within the pore, while signal element 4b is outside the pore (FIG. 6 at Point “B”). Yet because no analyte is present in the test sample, no binding between the analyte and analyte ligand 2 occurs at point “C”. And as the polarity of the voltage is changed as described herein, the analyte detection complex 1 is pulled out of the nanopore assembly at point “D” (FIG. 6), i.e., very early in the application of the second voltage. For example, because there is no analyte-ligand binding, the analyte does not prevent the analyte detection complex 1 from translocating back to the trans side of the nanopore (as compared to FIGS. 3-5). Hence, no binding signal is determined. Likewise, as the voltage opposite in polarity to the first voltage is further increased to point “E”, the nanopore remains open, with no dissociation voltage being determined (FIG. 6). Rather, an open channel signal on both the “positive” and “negative” can be detected.

In certain example embodiments, recycling a nanopore can be used to increase the confidence level of the analyte-ligand binding assessment of the nanopore. That is, in examples where the analyte dissociates from the analyte ligand 2, the same nanopore can be re-used multiple times as described herein to assess—and then re-assess—the interaction of the analyte with the analyte ligand 2. As such, recycling a nanopore can provide multiple data points for each nanopore assembly, hence providing additional information about analyte-ligand interactions.

Additionally or alternatively, in certain example embodiments multiple nanopore assemblies directed to the same analyte can be used on a chip to further increase the confidence of the analyte-ligand binding assessment. For example, each such nanopore assembly can be used to assess the analyte-ligand binding interaction and, when dissociation occurs, the multiple nanopores can also be recycled as described herein, thereby further increasing the confidence of the analyte-ligand binding assessment (via multiple nanopore and nanopore recycling). Thus, by increasing the number of nanopore assemblies directed to a given analyte—and by re-cycling a given nanopore assembly as described herein—the confidence of the analyte-ligand binding assessment can be substantially increased.

In certain example embodiments, subsets of different nanopore assemblies can be formed on single chip, with each individual subset directed to the same target analyte. Hence, in such embodiments a single chip can be used as described herein to assess binding interactions between different analytes and their respective ligands on the chip. Further, for each subset of nanopore assemblies, the confidence level of the analyte-ligand assessment can be increased as described herein, such as by increasing the number of nanopore assemblies in the subset and/or recycling of each nanopore assembly as described herein.

As those skilled in the art will appreciate, a variety of methods are available to differentiate among different nanopores populations on a chip. For example, different nanopore types, such as pores with smaller or larger pore sizes, can be used and readily differentiated based on techniques known in the art. With such configurations, for example, a nanopore with a larger opening can provide a larger current signal than a pore with a smaller opening, thus permitting differentiation of the pores on the same chip. The different nanopores can then be correlated with the analytes they are configured to detect, thus permitting identification of different analytes on the same chip. Other methods of differentiation include the blockade level of the analyte detection complex 1 as a whole and/or the threading element, the electrical signal associate with the pore in the absence of analyte, including the current-voltage profiles of the pores. In certain example embodiments, different nanopore assemblies can be differentiated using a control analyte. That is, a known analyte could be show identify a population of nanopore assemblies that bind the specific analyte. Using such methods, nanopore assemblies directed to analyte AA, for example, can be differentiated from nanopore assemblies directed to analytes BB or CC.

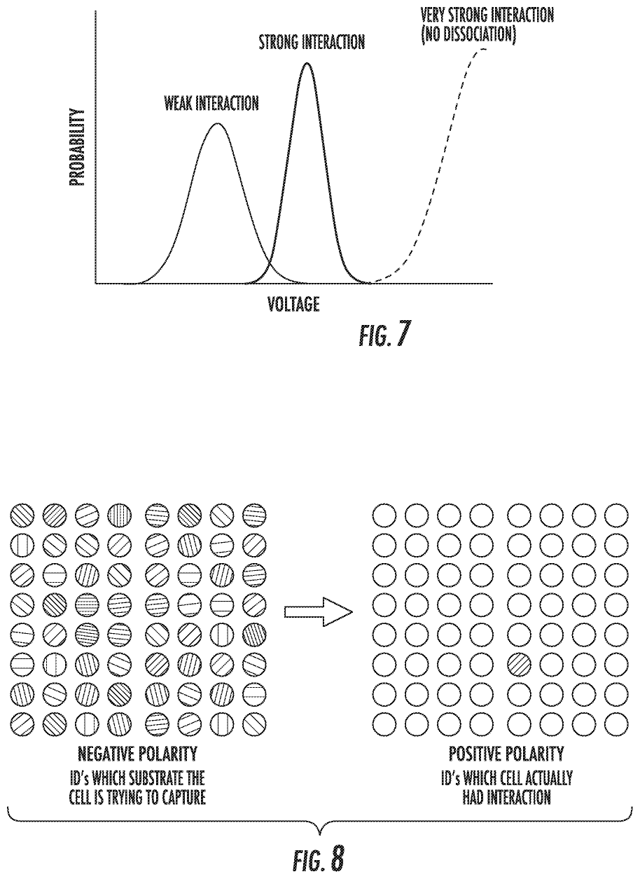

With reference to FIG. 7, provided is an illustration showing an example confidence level distribution of individual analyte captures and dissociations for weak, strong, and very strong analyte-ligand interactions, in accordance with certain example embodiments. In such example embodiments, the relative binding strengths among different analyte-ligand pairs on the same chip can be assessed and compared.

For example, for multiple nanopore assembly subsets—where each subset is directed to the same analyte but where the different subsets are directed to different analytes—the voltage level applied throughout a given binding-dissociation cycle can be plotted against the probability of analyte binding. The peaks, for example, correspond to dissociation of an analyte-ligand binding pair. For weak interactions, such as those illustrated in FIG. 3, a lower voltage is required for dissociation as compared to stronger binding interactions (FIG. 7). For strong interactions, such as those illustrated in FIG. 4, more voltage is required for dissociation (FIG. 7). And for very strong interactions, such as those shown in FIG. 5, no dissociation occurs despite a higher voltage (FIG. 7). The different voltages can then be compared, for example, thereby providing an indication of the relative binding strength of the different analyte-ligand pairs.

In certain example embodiments, the methods and systems described herein can be used to identify the detected analyte. For example, when an analyte is detected as described herein, such as via the binding signal, the specific identity of the analyte can be determined based on the known identity of the analyte ligand. If for example the analyte ligand 2 is a specific antibody, such as monoclonal antibody or functional fragment thereof, then detection of the antigen via the methods and systems described herein can be used to identify specific antigen found in the fluid solution. If the analyte ligand 2 is directed to a specific disease marker, such as a protein marker, the methods and systems described herein can be used to identify the specific marker as being present in a sample. Such embodiments are useful, for example, when analyzing a fluid sample from a subject for the presence of a particular analyte.

In certain example embodiments, the methods and systems described herein can be used on a single chip to detect and identify multiple known analytes on the same chip. Such embodiments are useful, for example, for analyzing a test sample for the presence (or absence) of multiple known analytes. As those skilled in the art will appreciate, current chip technology permits the deposition of hundreds of thousands of nanopores (or more) on a single chip. Hence, by using the methods and compositions described herein, thousands of different nanopore assemblies can be used on the same chip to test a fluid sample for thousands of different analytes.

For example, multiple subsets of nanopore assemblies can be assembled as described herein, with each subset being arranged to detect a different, known analyte. Each subset of nanopore assembly assemblies, for example, can include the same analyte ligand 2 and therefore be directed to the same known analyte, while different subsets are directed to different analytes. To distinguish among the different subsets of nanopore assemblies, each subset of nanopore assemblies, for example, can include a subset-specific signaling element. For example, one subset may have a specific signal element 4b that is different from another subset of nanopore assemblies that have a different signal element 4b. In certain example embodiments, the different subsets may be distinguishable based on the inclusion of an additional signal element, such as a third signal element. In other example embodiments, one subset of nanopore assemblies may include analyte detection complexes that have three signal elements associated therewith while other subsets may have four signal elements associated therewith. As those skilled in the art will appreciate, the different subsets of nanopore assemblies can be differentiated in many ways.

Once the different subsets of nanopore assemblies are assembled on the chip, the chip can be contacted with test sample as described herein, such as with a fluid sample from a subject. If any of the known analytes are present in the test sample, binding of the analytes to the analyte ligands can be assessed by switching the polarity of the voltage and determining a binding signal, as described herein. The binding of an analyte to an analyte ligand 2 can then be determined based on the binding signal. In other words, the binding signal provides an indication that the analyte is present in the test sample. In certain example embodiments, the binding strength of the different analyte-ligand pairs can also be assessed by continuing to increase the second voltage as described herein. Thus, when multiple analytes are analyzed on the same chip, not only are analyte-ligand pairs identified, but those with the strongest binding can also be identified.

Likewise, in certain example embodiments, a single chip can be used in the discovery of new analyte-ligand pairs. Such embodiments, for example, have many useful applications, such as in the areas of drug discovery and diagnostic reagent development. For example, different subsets of nanopore assemblies can be formed on a chip, with each subset including a different analyte ligand to an unknown ligand. Further, the nanopore assemblies can be differentiated as described herein. For example, nanopore assemblies that include analyte ligand X can be differentiated from nanopore assemblies that include analyte ligand Y or analyte ligand Z, as described herein. The nanopore assemblies can then be contacted with a test sample that contains several different candidate analytes to the ligands. Any binding of a candidate analyte to a particular ligand can then be determined as described herein. For example, certain analytes may bind only ligand X (and not other ligands). Further, of the analytes that bind ligand X, those with the strongest analyte-ligand binding can also be identified by increasing the second voltage as described herein.

With reference to FIG. 8, provided is an illustration showing the identification of specific analyte-ligand interactions on a chip, in accordance with certain example embodiments. As shown, multiple different nanopore assemblies are formed on a chip under a given first voltage, such as a negative polarity voltage (left panel). Based on signal data from the nanopores (in the open state) or from the nanopore assemblies, the different nanopore assemblies can be differentiated. As shown, different subsets of the same nanopore can be formed on the chip, as illustrated as shown in FIG. 8 (left side). After the nanopore assemblies are contacted with a test sample, a second voltage opposite in polarity to the first voltage is applied (e.g., a positive voltage) (FIG. 8 (right side)). As the second voltage is applied, any analyte-ligand binding pairs can be identified as described herein. As shown in FIG. 8, for example, a signal analyte-ligand interaction can be identified.

In still other example embodiments, the methods and systems described herein can be used to determine a dissociation constant between an analyte-ligand pair. For example, a dissociation voltage for the analyte-ligand pair can be obtained based on the dissociation signal. The dissociation voltage, for example, corresponds to the voltage at which the analyte-ligand dissociation occurs, which coincides with detection of the dissociation signal.

In certain example embodiments, to determine the dissociation constant, the dissociation voltage of the analyte-ligand pair can be compared to a predetermined reference dissociation voltage, which then allows identification of the dissociation constant for the analyte-ligand pair. The reference dissociation voltage, for example, corresponds to the voltage at which a known reference analyte-ligand pair dissociates when the reference analyte-ligand pair is subjected to the methods described herein. If a dissociation constant is known for the reference analyte-ligand pair, then the dissociation constant can be assigned to the analyte-ligand pair being tested. For example, the dissociation voltage for the analyte-ligand pair being examined can be matched to reference dissociation voltage, the matching dissociation voltage having an associated dissociation constant that can be assigned to the analyte-ligand pair being examined.

In certain example embodiments, the reference dissociation voltage can be obtained from a curve of dissociation voltages of control analyte-ligand pairs and their known dissociation constants. For example, nanopore assemblies with analyte ligands directed to different control analytes can be formed on a chip as described herein. In certain example embodiments, nanopore assemblies with analyte ligands directed to the analyte to be tested can also formed on the same chip. Thereafter, the chip is contacted with the control analytes and, in certain example embodiments, the analyte to be examined can also be applied to the chip (i.e., the test analyte). For example, in embodiments where the test analyte is to be tested on the same chip along with the control analytes, the control analytes and test analyte can be mixed together before the chip is contacted with the mixture.

After the chip is contacted with the mixture, the dissociation voltages for the control analytes can be determined as described herein, and a curve can be generated by plotting the dissociation voltages against the known dissociation constants for the control analyte-ligand pairs. By thereafter matching the dissociation voltage of the test analyte-ligand pair to a voltage on the curve (i.e., a reference dissociation voltage), a dissociation constant for the test analyte-ligand pair can be determined. In certain example embodiments, numerous cycles of binding and dissociation can be performed as described herein, thereby increasing the confidence level of the dissociation voltage determination—both for the test analyte-ligand pairs and any control analyte-ligand pairs.

In addition to detecting analyte binding and determining analyte-ligand binding strength, the methods and systems described herein can be used to determine the concentration of one or more analytes in a fluid solution that is applied to a chip. That is, analyte-ligand binding interactions can be assessed and identified as described herein, thereby allowing determination of the concentration of an analyte in solution. For example, multiple nanopore assemblies—each associated with an analyte detection complex directed to a specific analyte—can be formed on a chip as described herein. Likewise, nanopore assemblies directed to a control analyte can be formed on the chip. Thereafter, the chip including the nanopore assemblies can be contacted as described herein with one or more test analytes, along with a predetermined concentration of the control analyte—thus allowing the analytes to bind to their cognate analyte ligands 2. The second voltage opposite in polarity to the first voltage is then applied across the nanopore assembly until a binding signal is obtained, as described herein.

By counting the number of binding signals that are associated with the test analyte-ligand pairings on the chip, a binding count for the analyte-ligand pair can be determined. Hence, the binding count corresponds to the total number of analyte-ligand bindings that occur when the second voltage is applied across the nanopore assembly. In certain example embodiments, the confidence level of the binding count can be increased by cycling the test analyte-ligand pairs between bound and un-bound states as described herein (i.e., recycling the nanopores). For example, the binding count can correspond to the mean or median number of analyte-ligand bindings over multiple cycles of association and dissociation, as described herein.

In addition to determining the binding count for the test analyte-ligand pair, a reference count can be simultaneously determined for the control analyte-ligand binding pairs. The reference count, for example corresponds to the total number of control analyte-ligand bindings that occur when the second voltage is applied across the nanopore assembly. And like the test analyte-ligand pairs, the confidence level of the reference count can be increased by cycling the control analyte-ligand pairs between bound and un-bound states as described herein. For example, the reference count can correspond to the mean or median number of control analyte-ligand bindings over multiple cycles of association and dissociation, as described herein.

To determine the concentration of the test analyte in the solution, for example, the determined binding count can be compared to the determined reference count. As an example, if the control analyte is known to be present in a concentration of 10 μM when added to the chip, and the nanopore assemblies directed to control analyte bind an average of 1000 captures per cycle, the reference count would be 1000 for the 10 μM sample. If during the same set of cycles, for example, the average binding count for the test analyte was also 1000, then the concentration of the test analyte can be inferred to be 10 μM. But if the average binding count for the test analyte was 2000, i.e., twice as much as the control analyte, then the concentration of the test analyte would be 10 μM. Alternatively, if the if the average binding count for the test analyte was 500, i.e., half as much as the control analyte, then the concentration of the test analyte would be 5 μM.

In view of the many possible embodiments to which the principles of the disclosed invention may be applied, it should be recognized that the illustrated example embodiments are only preferred examples of the invention and should not be taken as limiting the scope of the invention. Rather, the scope of the invention is defined by the following claims. We therefore claim as our invention all that comes within the scope and spirit of these claims.

1. An analyte detection complex, the analyte detection complex comprising an analyte ligand, a threading element, a signal element, and an anchoring tag.

2. The analyte detection complex of claim 1, wherein the analyte ligand is located on a proximal end of the analyte detection complex, the signal element is associated with the threading element, and wherein the anchoring tag is located on the distal end of the threading element.

3. The analyte detection complex of claim 2, wherein the analyte ligand is an antibody or functional fragment thereof.

4. (canceled)

5. The analyte detection complex of claim 2, wherein the anchoring tag comprises a biotin tag.

6. The analyte detection complex of claim 2, wherein the signal element comprises an oligonucleotide sequence, a peptide sequence, or polymer.

7. The analyte detection complex of claims 6, wherein the signal element comprises an oligonucleotide sequence of about 40 nucleotide pairs.

8. The analyte detection complex of claim 7, wherein the oligonucleotide sequence comprises a series of T residues or a series of N3-cyanoethyl-T residues.

9. The analyte detection complex of claim 2, further comprising a second signal element.

10. The analyte detection complex of claim 9, wherein the second signal element comprises an oligonucleotide sequence, a peptide sequence, or polymer.

11. The analyte detection complex of claim 10, wherein the signal element comprises an oligonucleotide sequence of about 40 nucleotide pairs.

12. The analyte detection complex of claim 11, wherein the oligonucleotide sequence comprises a series of T residues or a series of N3-cyanoethyl-T residues.

13. A nanopore assembly comprising the analyte detection complex of claim 2.

14. The nanopore assembly of claim 13, wherein the nanopore assembly is a heptameric alpha-hemolysin nanopore assembly.

15. A method for assessing binding strength between an analyte and an analyte ligand, the method comprising:
providing, in the presence of a first voltage, a chip comprising a nanopore assembly according to 14, wherein the nanopore assembly is disposed within a membrane and wherein a sensing electrode is positioned adjacent or in proximity to the membrane;
contacting the chip with a fluid solution comprising the analyte, wherein the analyte comprises a binding affinity for the analyte ligand of the analyte detection complex;
applying an incrementally increased second voltage across the membrane, wherein the second voltage is opposite in polarity to the first voltage;
in response to applying the incrementally increased second voltage across the membrane, determining, with the aid of the sensing electrode, a binding signal, wherein the binding signal provides an indication that the analyte is bound to the analyte ligand; and
as the second voltage is further increased, determining, with the aid of the sensing electrode, a dissociation signal, wherein the dissociation signal provides an indication of the binding strength between the analyte and analyte ligand.

16. The method of claim 15, wherein the first voltage across the membrane positions the analyte ligand on a cis side of the membrane.

17. The method of claim 16, further comprising determining, with the aid of the sensing electrode, a threading signal, wherein the threading signal provides an indication that the threading element is located within the pore of the nanopore assembly.

18. The method of claim 17, further comprising comparing the threading signal to the binding signal, wherein the comparison provides the indication that the analyte is bound to the analyte ligand.

19. The method of claim 18, further comprising determining, from the dissociation signal, a dissociation voltage associated with dissociation of the analyte from the analyte ligand.

20. The method of claim 19, further comprising comparing the determined dissociation voltage with a reference dissociation voltage.

21. The method of claim 20, further comprising determining, from the comparison of the determined dissociation voltage to the reference dissociation voltage, a dissociation constant for the analyte and analyte ligand binding pair.

22. A method of determining concentration of an analyte in a fluid solution, comprising:
providing, in the presence of a first voltage, a chip comprising a plurality of nanopore assemblies according to 14, wherein the nanopore assemblies are disposed within a membrane and wherein at least a first subset of the nanopore assemblies comprise a first analyte ligand;
positioning a plurality of sensing electrodes adjacent or in proximity to the membrane;
contacting the chip with a fluid solution comprising a first analyte, wherein the first analyte comprises a binding affinity to the first analyte ligand;
determining, with the aid of the plurality of sensing electrodes and a computer processor, a binding count, wherein the binding count provides an indication of the number of binding interactions between the first analyte ligand and the first analyte;
comparing the determined binding count to a reference count;
determining, based on the comparison of the binding count to the reference count, a concentration of the analyte in the fluid solution.

23. The method of claim 22, wherein determining the binding count comprises:
determining, with the aid the plurality of sensing electrodes and for each nanopore assembly of the first subset of nanopore assemblies, a threading signal, wherein the threading signal provides an indication that the threading element is located within the nanopore of the nanopore assembly;
applying an incrementally increased second voltage across the membrane, wherein the second voltage is opposite in polarity to the first voltage;
in response to applying the incrementally increased second voltage across the membrane, determining, and with the aid of the plurality of sensing electrodes and for each nanopore assembly of the first subset of nanopore assemblies, a binding signal;
comparing, for each nanopore assembly of the first subset of nanopore assemblies, the determined threading signal with the determined binding signal, wherein the comparison provides an indication that the first analyte is bound to the first analyte ligand; and;
determining, from the comparison of each of the determined threading signals with the determined binding signals, a total number of indications that the first analyte is bound to the first analyte ligand, wherein the total number of indications corresponds to the binding count.

24. The method of claim 23, wherein the plurality of nanopore assemblies further comprises a second subset of nanopore assemblies, wherein each of the nanopore assemblies of the second subset comprises a second analyte ligand, the second analyte ligand comprising a binding affinity to a control analyte.

25. The method of claim 24, further comprising determining the reference count, wherein determining the reference count comprises contacting the fluid solution with a predetermined amount of the control analyte, thereby providing a predetermined concentration of the control analyte in the fluid solution.
